(19) United States
(12) Patent Application Publication
(10) Pub. No.: US 2021/0310976 A1
(43) Pub. Date: Oct. 7, 2021

(54) System For Slow Release Of Enzymes

(71) Applicant: Milo Sensors, Inc., Santa Barbara, CA (US)

(72) Inventors: Peter Lamberg, Santa Barbara, CA (US); Bob M. Lansdorp, Santa Barbara, CA (US)

(21) Appl. No.: 17/301,440

(22) Filed: Apr. 2, 2021

Related U.S. Application Data
(60) Provisional application No. 63/004,980, filed on Apr. 3, 2020.

(51) Int. Cl. G01N 27/327; B01J 2/28; C12N 9/04; B01J 31/00

(52) U.S. Cl. CPC G01N 27/327; B01J 31/003; C12N 9/0006; B01J 2/28

(57) ABSTRACT

Maintaining functionality in a device reliant on active biocatalysts. A water-impermeable container has one or more biocatalysts mixed and packed with stabilizers to function as a slow-release device. Depending on its adjustable geometries the device will provide a modifiable trickle of biocatalysts into a liquid released from a dry and stable state.

This invention was made with government support under AA 026125, awarded by National Institute of Health (NIH). The government has certain rights in the invention.

This application claims priority from provisional No. 63/004,980, filed Apr. 3, 2020, the entire contents of which are herewith incorporated by reference.

### BACKGROUND

Biological catalysts such as enzymes in electrochemical devices can be highly specific and potentially very effective catalyzers of nearly any analyte.

Stability is often a bottleneck to sustained performance in applications of bio-powered technology. Once a biological unit, such as an enzyme, is put into solution, it will start to naturally decompose.

A common way to keep such units fresh is to refrigerate, freeze or lyophilize batches and dissolve them immediately before use. Indeed, most biological assays and test kits have temperature sensitive biological components.

In a freestanding device, however, it is usually not possible to maintain a low-temperature compartment for storing unused active compounds.

A most common portable enzymatic devices on the market include glucose monitors powered by glucose oxidase (GOx). While some progress has been made to stabilize GOx for a few weeks, the measurement device needs to be supplied with fresh preparations of enzyme intermittently. This is commonly done using interchangeable cassettes or chips containing fresh enzyme. However, when using biological components with much shorter hydrated lifetime frequent cassette exchanges makes the device too burdensome to use.

In a state-of-the-art wearable alcohol biosensor (Lansdorp et al, Sensors, 2019), enzyme was fully hydrated within a short period of time, and the sensor was observed to stop functioning after 24 hours, due to the loss of enzyme activity.

Prior art makes mention of open-form cylindrical devices for controlled drug release (U.S. Pat. No. 5,851,547A), however this prior art fails to consider the application of catalysts to a biosensor, where extended release of catalysts can improve biosensor lifetime.

Prior art also mentions the use of immobilization protocols and molecular modulation to improve the longevity of a sensing device using enzymes, most often including the use of aldehydes and other crosslinkers (US20170009270A1). However, many biocatalysts are either inhibited by such modifications or completely deactivated. Therefore, what is desired is a means of extending the operational life of enzyme-based devices without the use of harmful chemicals.

### SUMMARY

Therefore, the inventors recognize the need for an integrated solution to replenish active biological components to a bioelectronic device. Having an integrated system is especially useful in wearable monitoring devices.

The present invention describes structure and methods for the slow and sustained release of catalysts from an isolated compartment.

This invention describes a small integratable compartment with stabilized biological components that are slowly added to a solution over time.

### BRIEF DESCRIPTION OF THE DRAWINGS

FIG. 1 shows a schematic representation of the cylindrical container with a biocatalyst resin;

FIG. 2 shows a wearable platform consisting of a cartridge containing a slow-release mechanism and a biosensor;

FIG. 3 shows a Schematic of a slow release mechanism with two biocatalyst resin phases (Φ1, Φ2) and the anticipated dispersion dynamics;

FIG. 4 shows a photographs of biocatalyst resin deposits with varying degrees of dispersion, with and without contrast enhancement;

FIG. 5 shows a Graph of biocatalyst dispersion versus time of a one-phase biocatalyst mixture;

FIG. 6 shows a Flowchart of the assembly and integration process;

FIG. 7 show Schematic of biological catalysts and stabilizers compressed into a rod; and

FIG. 8 shows a Schematic illustration of a method for loading of a cylindrical container.

### DETAILED DESCRIPTION

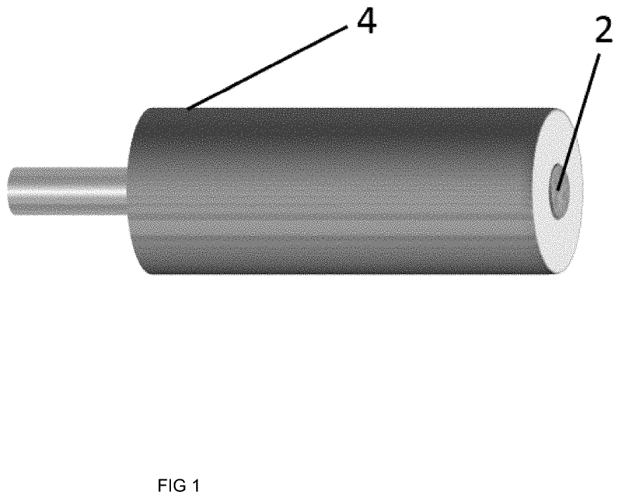

FIG. 1 illustrates an embodiment where a dry biocatalyst 2 is packed into a cylindrical container 4 which acts as a biocatalyst well. This cylindrical container 4 houses the biocatalyst 2 that can subsequently be placed into a liquid to hydrate the biocatalyst.

In another embodiment, the shape of the cylindrical container biocatalyst well has a radius that varies with length such as a cone. In another, the biocatalyst well is not cylindrical, but rectangular, or other geometric shapes. The container itself is liquid impermeable, but has an opening with a specified geometry. The container can be formed of plastic, rubber, glass, or metal, or other materials known to those of ordinary skill in the art. The opening is used, as described herein, to expose to the biocatalyst to liquid at a controlled rate.

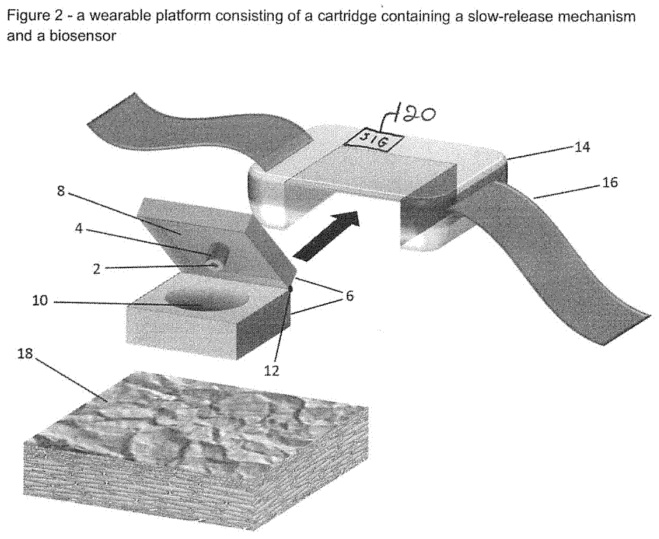

In another embodiment, shown in FIG. 2, a container 4 with dry catalyst 2 is combined with an electrochemical sensor such as a continuous wearable alcohol sensor. A cartridge 6 has two compartments: a dry compartment 8 and liquid compartment 10. In the dry compartment 8, a cylindrical container 4 made of polypropylene contains lyophilized Alcohol Oxidase (AOD) 2 as a biocatalyst. The liquid compartment can include water or a buffer or other liquid. The buffer can be for example a phosphate buffered saline.

The compartments may be sealed using heat-sealable films, adhesive films, or other water-impermeable materials or peelable seals familiar to those skilled in the art.

In this pre-activated configuration, where the dry is separate from the wet, the cartridge is stable for 6 months.

To activate the cartridge, the cylindrical container 4 can be immersed into the liquid compartment 10 containing a buffer, by folding together the two compartments 8 and 10 by means of a hinge 12. The cartridge 6 can be attached to a wristband housing 14 that connects to a strap 16. Once the 2 compartments have been folded together, the liquid starts the diffusion process and activates dissolved biocatalyst, allowing the cartridge to carry out a chemical reaction. A signal measuring device 120 on the wearable platform measures the signal induced by the chemical reaction using the cartridge and its reaction with analytes diffusing through the skin 18. The signal measuring device 120 can use amperometric measurements through cartridge electrodes. The signal measuring device 120 can communicate the signal wirelessly, for example, to a receiver. Thus, the cartridge can be activated by folding together the 2 parts 8 and 10 which thus exposes the cartridge to the liquid, allowing at that point transdermal sensing of alcohol non-invasively after strapping the embodiment on skin 18.

The release rate of fresh enzymes from the device is tuned by the opening size, as well as by other techniques described herein, to match the deactivation rate of the enzymes in the liquid compartment. This embodiment enables alcohol oxidase to be slowly added to the liquid compartment, extending the lifetime of the cartridge over those in the prior art, for example from 24 hours of continuous sensing up to 72 hours or more of continuous sensing, a substantial improvement over prior art.

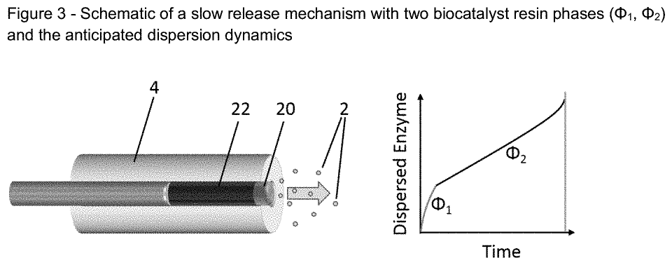

In another embodiment, container 4 can be loaded with more than one type of resin, for example with biocatalyst and stabilizer, as seen in FIG. 3. The resins can be loaded into container 4 in succession using pre-cut deposits. If a higher initial dispersion rate is desired, a short resin with more rapid hydration dynamics 20 can be placed in front of a larger resin deposit 22, and vice versa. Thus, the dispersion rate can be adjusted into stages (Φ) by means of phased resins with different stabilizer compositions. Those stages can form deposits that have phases of dissimilar solubility in the liquid, thus allowing the catalyst to dissolve at different levels during the different phases.

Alternatively, the rate of dispersion can be adjusted using a cylinder that varies by length, such as a cone. A widening diameter will increase dispersion rate due to an increase in exposed surface area of the resin. Similarly, a narrowing diameter will decrease the dispersion rate. Adjusting dispersion rate has the advantage of enabling a device to quickly fill with a biocatalyst to reach a saturated condition, and maintain saturation for an extended period of time.

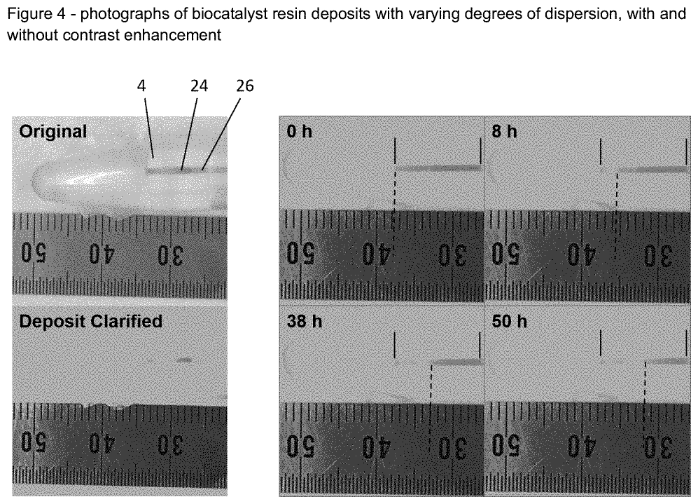

In one embodiment, shown in FIG. 4, the biological catalyst enzyme alcohol oxidase (AOD) 2 is stabilized with polysaccharides and lyophilized (Sun Chemical). The lyophilized powder was thawed and compressed into pliable resin 24 after exposure to ambient humidity. The resin was manipulated into a rod shape to fit into the geometry of a clear borosilicate glass cylinder 4 with an inner diameter of 0.6 mm and an adjustable length between 1-20 mm by means of a slidable metal stopper 26. This has the effect of embedding the catalyst into the container. Packing of resin 24 into cylinder 4 could alternately be accomplished using a sharp metal tweezer cleaned and dried before use, or other means familiar to those skilled in the art to embed the catalyst. Alternately, the resin could be replaced with powders, other phases of matter familiar to those skilled in the art. The resin can be compressed between metal stopper 26 and a second flat surface to ensure that no air gaps are present. Alternatively, other means of compression can be used, such as humidity-controlled compressed air or by means of automated pistons.

Alternatively, the catalyst can be embedded using the water-soluble material that has one or more compounds of limited solubility. The compounds of limited solubility can be an organic or inorganic wax, a cellulose material, or starch material, in embodiments.

For the geometry described above, the transparent cylinder 4 containing resin 24 was placed in a transparent eppendorf tube with 1 mL PBS containing 0.25% agarose gel and sealed. To track the interface, a time-lapse photographic series was taken, with intervals of 10 minutes, starting at the time the loaded container 4 was submerged into the liquid (see FIG. 4). The position of the solid/liquid interface was visually observed and recorded as a function of time. The photograph color and contrast was adjusted to enhance the visibility of the solid/liquid interface, as seen in the left side of FIG. 4. In the 4 inlay images on the right side of FIG. 4, contrast-adjusted images are shown at four representative times with dashed lines tracking the interface.

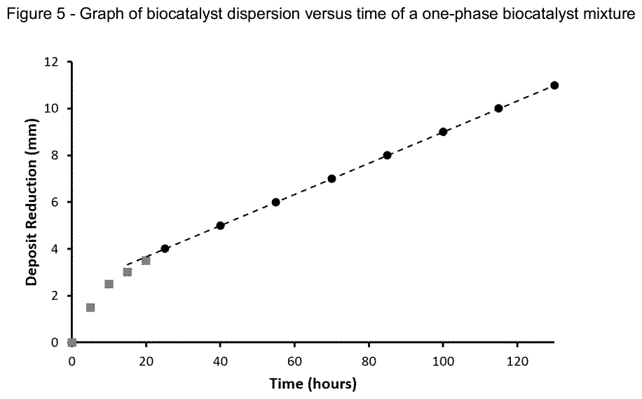

The results for the embodiment shown in FIG. 4 are plotted in FIG. 5. Over the course of 130 hours, the 11 mm AOD deposit had completely dispersed. The interface is observed to slowly recede, releasing a steadily increasing amount of enzyme into solution.

Dispersion rate of any mixture of biocatalysts and stabilizers can be measured by means of a visual tracking of the receding deposit-liquid interface. Thus, the dispersion rate for a specific container geometry can be estimated as follows:

$${σ}_{resin} = - \frac{Δ d}{Δ t}$$

Where σᵣₑₛᵢₙ is the resin dispersion rate in mm/day and Δd/Δt is the receding interface in millimeters per unit time. The rate of increase is initially high, and then reaches a steady state rate of dispersion of 1.6 mm/day. In contrast, the same amount of lyophilized and stabilized enzyme mixed directly into the liquid disperses within seconds. This demonstrates a reduction to practice of a diffusion controlled release of alcohol oxidase enzyme into a liquid solution.

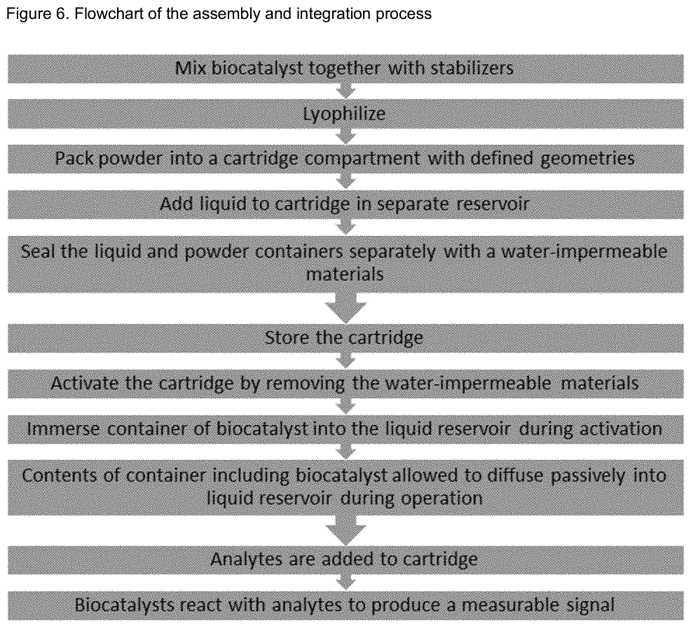

The general assembly process flowchart of an embodiment integrated in a biosensor is shown in the flowchart of FIG. 6. The stabilizer and biocatalyst mixture may or may not be mixed and lyophilized before insertion into the biosensor cartridge. The cartridge is sealed using a water-impermeable material, and allowed to be stored for up to 6 months on a shelf at room temperature. The cartridge is activated by removing the water impermeable materials, for example by peeling a heat-sealed foil, or puncturing a metalized foil. Next, the biocatalysts contained in the cartridge container are immersed into the liquid reservoir in the cartridge. In a preferred embodiment the biocatalyst mixture is allowed to diffuse passively, but can alternatively be sped up intermittently by liquid agitation, temperature adjustments or by similar means. The liquid in compartment 4 may also be added at the time of cartridge activation.

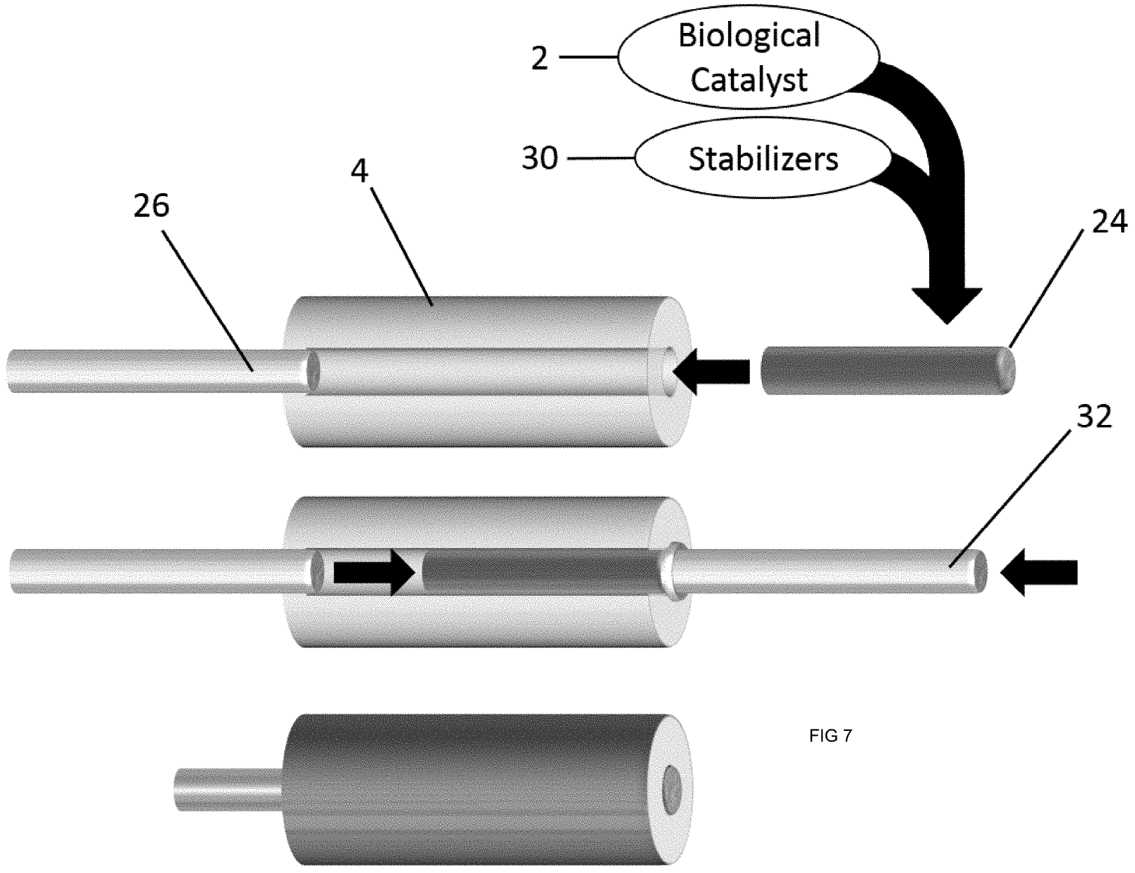

FIG. 7 schematically illustrates a method for packing biocatalyst resin in a cylindrical container. Resin 24 can be made pliable enough to be physically compressed into a geometry that fits container 4 by adjusting the stabilizer mixture 30, which is mixed with a biological catalyst 2. Resin 24 is inserted into container 4 by manipulation with tweezers, or other tools familiar to those skilled in the art. Slidable stopper 26 is braced against a metal bracer 32, thus compressing resin 24 into a dense deposit. Alternatively, the biocatalyst may be a dry powder that is compressed into a pellet, or other physical forms familiar to those skilled in the art.

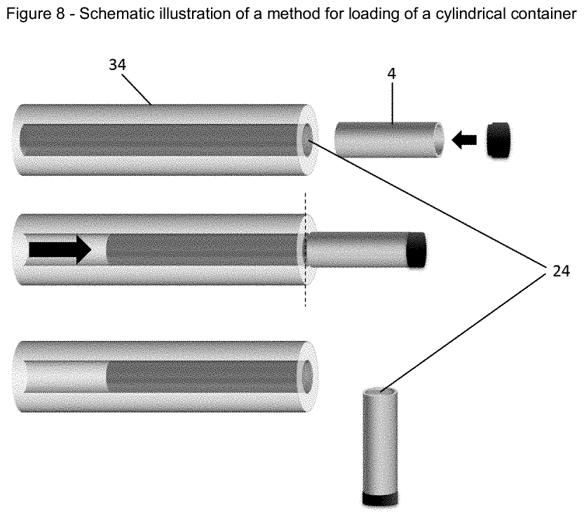

FIG. 8 illustrates a way of quickly loading smaller containers with resins. Resin transfer can be done by using a transfer capillary 34 with the same inner diameter as container 4. A resin deposit 24 can be packed into the transfer capillary by the means described in FIG. 7. The shaped resin deposit 24 is pushed from capillary 34 into container 4 by means of, for instance a piston, air pressure or similar methods of physical manipulation. Once the desired length of deposit has been inserted the resin can be severed, for instance by means of a razor blade.

Once loaded, the exposed end of the device can be covered by a plastic cap, metallized film, water impermeable material, or other materials familiar to those skilled in the art. Container 4 may also be formed by injection molding of plastic, machining of metals by drill, or other forms familiar to those skilled in the art.

Although only a few embodiments have been disclosed in detail above, other embodiments are possible and the inventors intend these to be encompassed within this specification.

The previous description of the disclosed exemplary embodiments is provided to enable any person skilled in the art to make or use the present invention. Various modifications to these exemplary embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A device for slow and controlled release of catalyst into a solution comprising:
a liquid impermeable container with an opening, the opening having a specified geometry;
a catalyst, located in the container and packed in the liquid impermeable container; and
a liquid, which is exposed to the catalyst at a specified rate which is dependent on at least the specified geometry.

2. The device of claim 1, where the catalyst is a biological enzyme.

3. The device of claim 1, where the catalyst is alcohol oxidase.

4. The device of claim 1, where the liquid is composed primarily of water.

5. The device of claim 1, where the liquid is a phosphate buffered saline.

6. The device of claim 1, where the container is comprised primarily of a material from the group consisting of plastics, rubber, glass, or metal.

7. The device of claim 1, wherein the container is initially sealed, and is exposed to the liquid at the specified rate at a time when the device is first used.

8. The device of claim 1, further comprising a water soluble material embedding the catalyst, where the water soluble material is a mixture containing at least one polysaccharide.

9. The device of claim 1, further comprising a water soluble material embedding the catalyst, where the water soluble material also contains one or more compounds of limited solubility, including but limited to:
An organic or inorganic wax
Cellulose, or
Starch.

10. The device of claim 1, where the catalyst is embedded in deposits that have phases of dissimilar solubility in the liquid and the phases of dissimilar solubility also affect the specified rate.

11. The device of claim 1, where one or more soluble materials are added, and where the catalyst is lyophilized after the soluble materials are added.

12. A biosensor comprising:
a cartridge with a dry compartment, and having a biocatalyst in said dry compartment;
a liquid reservoir holding a liquid;
a means of releasing the biocatalyst at a controlled rate into the liquid reservoir;
a housing, holding the cartridge; and
a signal measuring device, which measures a signal caused by a chemical reaction in the cartridge.

13. The biosensor of claim 12, where the sensor is used for continuous transdermal alcohol sensing, and the signal measuring device senses a signal that represents transdermal alcohol sensing.

14. The biosensor of claim 13, where the biocatalyst is Alcohol Oxidase enzyme.

15. The biosensor of claim 14, where the liquid in the liquid reservoir consists primarily of water.

16. The biosensor of claim 12, where the signal measuring device uses amperometric measurement through electrical contacts.

17. A method of extending an operational lifespan of a biosensor, comprising:
embedding enzyme and stabilizers into a cylindrical tube;
sealing at least one side of the cylindrical tube;
subsequently, at a time of desired use, removing the seal and after removing the seal, adding an unsealed side of the cylindrical tube to a liquid, to cause the enzyme and stabilizers to become hydrated by the liquid; and
using the enzyme which has been hydrated by the liquid to carry out a biosensing measurement.

18. The method of claim 17, wherein the biosensor is a wearable enzymatic alcohol sensor which measures alcohol as its biosensing measurement.

19. The method of claim 17, wherein the biosensor has an extended operational lifespan of at least 24 hours.
